(19) United States
(12) Patent Application Publication
(10) Pub. No.: US 2016/0166340 A1
(43) Pub. Date: Jun. 16, 2016

(54) Surgical Instrument

(71) Applicant: KUKA ROBOTER GMBH, Augsburg (DE)

(72) Inventor: Sven Brudniok, Langerringen (DE)

(21) Appl. No.: 14/908,658

(22) PCT Filed: Jul. 11, 2014
(86) PCT No.: PCT/EP2014/001908

§ 371 Date: Jan. 29, 2016

(30) Foreign Application Priority Data
Aug. 1, 2013 (DE) 10 2013 012 802.1

(51) Int. Cl. A61B 34/30; A61B 17/00

(52) U.S. Cl. CPC A61B 34/30; A61B 17/00234; A61B 2017/00327

(57) ABSTRACT

A surgical instrument has a pre-tensioned pair of flexible links comprising a first flexible link strand and a counter-rotating second flexible link strand for actuating a degree of freedom of the instrument, in particular an end effector, in opposite senses, by an input assembly. The first flexible link strand has a higher tensile stiffness than the second flexible link strand.

### TECHNICAL FIELD

The present invention relates to an in particular robot-guided, surgical instrument, a surgical robot with such an instrument, and a method for extending and actuating such an instrument at a distance from the body.

### BACKGROUND

In particular during robot-supported, minimally invasive surgery, instruments are frequently actuated by counter-rotating flexible link pairs.

For instance, a jaw of a vein clamp coupled with a drive sheave can be rotated by actuating two draw strands that wind around the drive sheave in opposite senses to close and/or open the vein clamp.

The draw strands are pre-tensioned to prevent a sagging of the draw strands and slippage of the drive sheave. However, this pre-tension places a load on the draw strands and bearings and limits the maximum force that can be applied by the jaw, wherein an anti-parallel force-pair, e.g. a torque, is also generalized herein as a force for purposes of a more compact specification.

### SUMMARY

One task of an embodiment of the present invention is to provide an improved, in particular robot-guided, surgical instrument, and/or to improve the operation of same.

According to one aspect of the present invention, a surgical instrument has one or several pre-tensioned pairs of flexible links, each with a first flexible link strand and a counter-rotating second flexible link strand for actuating a degree of freedom of the instrument in opposite senses with an input assembly.

A flexible link can in particular be configured as a rope and/or cable or as a band and/or belt, in particular as a flat, V, and/or ribbed band and/or belt. In one embodiment, it can contain plastic and/or metal, in particular can consist of these.

A flexible link strand is referred to herein in particular as the (free) section of a flexible link between an input member of the input assembly and an output member of an output assembly, in particular an end effector output assembly.

In one embodiment, a flexible link is permanently or removably secured in axial direction—in relation to a length axis of the flexible link—to the input and output member, in particular in a friction, substance-to-substance, or positive lock. In this case, the flexible link and/or its section between the mounting locations on the input and output member then represents the flexible link strand as defined by the present invention. In another embodiment, the flexible link wraps around the input and output member in a friction lock, in each case at least partially. In this case, a section of the flexible link between the inbound and/or outbound side from the and/or to the input and output member then in each case represents a flexible link strand as defined by the present invention. In this regard, a flexible link strand as defined by the present invention can equally designate a fixed region of a (secured) flexible link or an adjustable region of an (inbound and/or outbound) flexible link. In one embodiment, a flexible link open on one side at least partially wraps around the input member with a friction lock, wherein its two ends are each secured on the output member on the opposing side. In this case, a section of the flexible link between the inbound and/or outbound side from the and/or to the input member and the mounting location on the output member in each case represents a flexible link strand as defined by the present invention. In another embodiment, a flexible link open on one side conversely partially wraps around the output member with a friction lock, wherein its two ends are each secured on the input member on the opposing side. Then a section of the flexible link between the inbound and/or outbound side from and/or to the output member and the mounting location on the input member in each case represents a flexible link strand as defined by the present invention.

Accordingly, in one embodiment, the first flexible link strand and the second flexible link strand of one or several pairs of flexible links can each be attached on an input and/or output member connected together on the outbound side, in particular on the end effector side and/or on the input side, or separately from each other. One or several pairs of flexible links can accordingly be configured closed and/or ring-shaped, semi-open and/or connected on one side, or as two separate flexible links.

In one embodiment, the first and second flexible link strand are actuated by the same input member and/or actuate the same output member. For instance, they can wrap around a drive sheave or be secured on two extensions of a link arm. In the same manner, the first and second flexible link strand can be actuated by separate input members and/or actuate separate output members, wherein in one implementation the separate input members of the input assembly and/or the separate output members of the output assembly are synchronized in opposite senses preferably, in particular mechanically, hydraulically, pneumatically and/or by control technology. For instance, two linear actuators synchronized in opposite senses can retract and/or extend the first and second flexible link strand in opposing senses, or two flexible link ends can move two couplings of a mechanism, for instance of a parallelogram mechanism, in opposing senses. In one embodiment, one or several guide and/or coupling members, in particular discs, that guide and/or couple the flexible link strands can be arranged between the input assembly and the output assembly.

The degree of freedom actuated by the pair of flexible links in opposing senses can in particular be a degree of freedom, in particular a translational or rotational degree of freedom, of an end effector of the instrument, e.g. the rotation of a scalpel, a hook or a jaw of a clamp, scissors, pliers, etc. Accordingly, the output assembly can be an end effector assembly, and the output member and/or the output members can be coupled, in particular permanently connected, with the single or multi-part end effector. In the same manner, the degree of freedom can also be an articulated degree of freedom of an articulated instrument shaft or the like. Accordingly, the output assembly can be an instrument output assembly, and the output member and/or the output members can be coupled with the articulated instrument shaft. In one embodiment, the degree of freedom is a swivel degree of freedom of an end effector relative to an instrument shaft on which the end effector is seated in a movable and/or articulated manner, or a swivel degree of freedom of an instrument shaft portion relative to another instrument shaft portion on which the end effector is seated in a movable and/or articulated manner.

In one embodiment, several counter-rotating pairs of flexible links are arranged to actuate two or more degrees of freedom of the instrument, in particular an end effector, in opposing senses, wherein the present invention is described as follows in detail based on one pair of flexible links for purposes of a more compact specification.

FIG. 1—described in detail below—shows a static equilibrium between an input force F_{A} on an input member A and a reactive moment M_{R} acting on an output member 3 based on a contact of an end effector 3.1.

Input and output members are coupled through a pair of flexible links with a first flexible link strand 1 and a counter-rotating second flexible link strand 2 that are each pre-tensioned by a pre-tensioning device so that they have tensile pre-tension and/or tensile pre-tension force F_{V} in the unactuated state.

Due to the input force F_{A} resp. the reactive moment M_{R}, the tensile stress and/or the tensile force F₁ in the pulling first flexible link strand 1 is increased by an elastic portion (Δl*c₁) determined from the additional elongation Δl and the tensile stiffness c₁ of said flexible link strand:

F₁=F_{V}+Δl*c₁ (1)

Accordingly, the tensile stress and/or cut force F₂ is reduced in the yielding second flexible link strand 2 by the elastic portion (Δl*c₂) determined from the counter-rotational shortening Δl and the tensile stiffness c₂ of this flexible link strand:

F₂=F_{V}−Δl*c₂ (2)

The reactive moment M_{R} is derived from the static moment equilibrium with these two cutting forces F₁, F₂ and the lever arm r as:

M_{R}=(F₁−F₂)*r (3)

and with equation (1), (2) as

M_{R}=(c₁/c₂+1)*(c₂*Δl)*r (4)

The maximum reactive moment M_{R, max.} and therefore the maximum force that can be applied by the end effector on the environment and/or the maximum transferrable input force F_{A, max.} is derived in the yielding second flexible link strand 2 at vanishing tensile stress and/or cut force F₂=0:

F_{V}=Δl*c₂ (5)

inserted into (4) results in

M_{R, max}=(c₁/c₂+1)*F_{V}*r (7)

One recognizes that as the ratio of the tensile stiffness between the first and second flexible link strand increases while the pretension of the pair of flexible links remains the same, the maximum reactive moment M_{R, max} and therefore in particular a maximum force that can be applied by an end effector on the environment can be increased, and/or inversely, that only a lower pretension F_{V} is required for the same reactive moment. Since the pretension in particular places a load on the flexible link but also on the bearings, and is difficult to generate, increasing the ratio of the tensile stiffnesses can therefore advantageously result in an improved instrument, in particular by extending its service life due to the reduced load on bearings and flexible link and/or by improving its operation due to an increased maximum reactive moment and therefore in particular an increased force that can be applied by an end effector on the environment.

According to one aspect of the present invention, the first flexible link strand of at least one pretensioned pair of flexible links, preferably of two or more, in particular of all pretensioned pairs of flexible links of the surgical instrument, respectively exhibits a larger tensile stiffness than the second flexible link strand of this pair of flexible links.

As is derived in particular from equations (1), (2), tensile stiffness c herein in particular is defined as the relationship and/or ratio of a tensile force F on the flexible link strand and/or a tensile stress in the flexible link strand to a resulting effective and/or functionally applying elongation Δ| and/or increase in stretch (Δl/l) of the flexible link strand (c=F/Δl), preferably absolute or specific and/or in relation to the length | of the flexible link strand (c=F/(Δl/l). An otherwise equal, twice as long flexible link strand therefore has a smaller absolute tensile stiffness as defined by the present invention (since both halves each elongate, thus resulting in a greater total elongation Δl), but has the same specific tensile stiffness, and/or the same tensile stiffness in relation to the length as defined by the present invention.

If the tensile stiffness for instance changes as a function of an increase in stretch and/or tensile stress, in one embodiment the tensile stiffness of the first flexible link strand at least for tensile and/or cutting forces in the flexible link strands is larger than the tensile stiffness of the second flexible link strand, where said forces are at least 1N and/or a maximum of 500N, in particular a maximum of 100N, in particular at least 10N, i.e. in particular at least within a normal working range. In one implementation, the tensile stiffness of the first flexible link strand is always greater than then tensile stiffness of the second flexible link strand. It is understood that normal operating conditions are referenced for the tensile stiffness. Accordingly, in one embodiment, for temperatures that range between 10 degrees Celsius and 40 degrees Celsius, the tensile stiffness of the first flexible link strand is greater than the tensile stiffness of the second flexible link strand.

It has been shown that the tensile stiffness of the first flexible link strand advantageously is at least 10 percent, in particular at least 25 percent and preferably at least 50 percent greater than the tensile stiffness of the second flexible link strand.

The tensile stiffness of a flexible link strand depends in particular on its material, in particular on its coefficient of elasticity and/or its modulus of elasticity. Accordingly, in one embodiment, a material of which the first flexible link strand consists wholly or at least partially has a greater tensile stiffness, in particular a greater modulus of elasticity, than a material of which the second flexible link strand consists wholly or at least partially. In this case, the first and second flexible link strand at least essentially can have the same geometry, in particular the same cross-sections, which can in particular simplify handling. In one embodiment, a material of which the first flexible link strand consists wholly or at least partially has a tensile stiffness, in particular a modulus of elasticity, that is at least 10 percent, in particular at least 25 percent and preferably at least 50 percent greater than the tensile stiffness and/or the modulus of elasticity of a material of which the second flexible link strand consists wholly or at least partially.

The tensile stiffness of a flexible link strand depends in particular on its geometry, in particular on its cross-section area. Accordingly, in one embodiment, the first flexible link strand—based on its geometry, in particular based on its cross-section—has a greater tensile stiffness than the second flexible link strand. In particular, the first flexible link strand can have along its entire length or at least along a section thereof a larger, in particular minimum, cross-section than the second flexible link strand. The first and/or second flexible link strand can each have one or multiple strands. Accordingly, in one embodiment, the first flexible link strand can have more, in particular equal, strands than the second flexible link strand. If the geometry-based tensile stiffness of the first flexible link strand is greater, the first and second flexible link strand, at least essentially, can have the same material, which in particular can simplify manufacturing. In one embodiment, the first flexible link strand has a minimum cross-section area, which is at least 10 percent, in particular at least 25 percent and preferably at least 50 percent greater than a minimum cross-section area of the second flexible link strand.

The two aforementioned aspects can be combined with each other. In particular, the first flexible link strand can throughout or at least in sections have a material with a greater modulus of elasticity and a greater cross-section area, in particular strand count, than the second flexible link strand.

In one embodiment, the flexible link strands are throughout made of the same material and have a constant cross-section area. In the same manner, the first and/or second flexible link strand can have different materials and/or geometries, even in sections. Mechanically, this corresponds to an in-series arrangement of springs and/or tensile stiffnesses. Accordingly, the tensile stiffness of the first flexible link strand can be increased by at least one section with a locally higher tensile stiffness, in particular made of a material with a locally greater modulus of elasticity and/or a locally greater cross-section area, and/or the tensile stiffness of the second flexible link strand can be reduced by at least one section with a locally lower tensile stiffness, in particular made of a material with a locally lower modulus of elasticity and/or a locally smaller cross-section area. Such a section can be spaced in particular from both ends of the particular flexible link strand, in particular attachments and/or the inbound and/or outbound regions, so that the first and second flexible link strands in one embodiment have matching end regions, which can in particular improve handling.

As discussed above, by increasing the tensile stiffness of the particular pulling flexible link strand and/or the load-strand of a pair of flexible links, the moment and/or force that can be applied at the same pretension can be increased, and/or the required pretension decreased. Many counter-rotating pairs of flexible links have a primary working direction, in which greater forces and/or moments are transferred than in the opposite direction. For instance, greater forces and/or moments are regularly required to close jaws on pliers, scissors, clamps, or the like, than to open these. In the same manner, larger forces and/or moments are regularly required to feed a blade, in particular a scalpel, in the direction of their cutting edge and/or for cutting, than to lift and/or release these. Accordingly, in one embodiment, the first flexible link strand that has the greater tensile stiffness is arranged to actuate the degree of freedom in a primary working direction, in particular for cutting or closing. A primary working direction is in particular defined as the direction in which the greater forces and/or moments are (to be) applied during operation. In one embodiment, the primary working directions of two pairs of flexible links, whose first flexible link strand respectively has a greater tensile stiffness, are arranged in opposite senses, as is in particular the case on two jaws of pliers, scissors, a clamp, or the like.

The pair of flexible links is pretensioned to avoid a slackening of secured flexible links and/or a slippage of wrapped-around flexible links. For this purpose, a pre-tensioning device is arranged in one embodiment to jointly pre-tension the first and second flexible link strand of one or several pairs of flexible links. In another embodiment, an in particular multi-part pre-tensioning device is arranged to separately pre-tension the first and second flexible link strands of one or several pairs of flexible links. A pre-tensioning device and/or a portion of a multi-part pre-tensioning device can in one embodiment have one or several mechanical, hydraulic, pneumatic, magnetic and/or electro-magnetic springs, wherein an (electro-)magnetic spring as defined by the present invention has two (electro-)magnetically opposing or attracting elements. In one embodiment, the pre-tensioning device is adjustable and/or configurable, for instance by changing a spring hardness, pre-tensioning length, or the like.

A surgical instrument can be actuated manually. In a preferred embodiment of an in particular robot-guided surgical instrument, the latter additionally or alternatively has an actuator assembly with one or several actuators. An actuator can in particular have one or several electro-motors. An actuator can be, in particular is, functionally connected with one or several input members of an input assembly of a pair of flexible links, preferably in a manner that the actuator actuates the first and second flexible link strand in a counter-rotating manner, in particular in opposing senses in a synchronized manner. The actuator can be, in particular is, directly or in particular through a gearbox functionally connected with the input assembly. The gearbox can in particular have a flexible link gearbox. Accordingly, an input member in one embodiment can be, in particular is, functionally connected with the output assembly through a pair of flexible links and with the actuator to an additional pair of flexible links.

As discussed in the introduction, an inventive instrument is in particular suited for minimally invasive surgery. Accordingly, it has in one embodiment an instrument shaft on which the one or several pairs of flexible links are arranged, wherein said instrument shaft is arranged for insertion into a natural or minimally invasive body opening, in particular a so-called trocar. The instrument can accordingly, in particular be an endoscope instrument with an end effector for insertion into a body, and an opposingly positioned, extra-corporal actuator assembly.

As is also discussed in the introduction, an inventive instrument is in particular suited for minimally invasive robotic surgery. Accordingly, in one embodiment, it has an interface for connecting to a robot. The interface can in particular be arranged on the instrument shaft and/or an actuator assembly. In one embodiment, the interface has a mechanical and/or magnetic, in particular electro-magnetic clutch to couple to a robot, and/or an interface to transmit signals, electrical energy and/or fluids, in particular gases and/or liquids, between the robot and the instrument.

According to one aspect of the present invention, a tensile stiffness of the first flexible link strand of one or several pre-tensioned pairs of flexible links is specified greater than a tensile stiffness of the second flexible link strand of the particular pair of flexible links, in particular by appropriately specifying a material and/or a geometry of the particular flexible link strand. According to another aspect of the present invention, a tensile stress increase in the first flexible link strand of one or several pre-tensioned pairs of flexible links resulting from an actuation of the input assembly of this pair of flexible links is greater due to its greater tensile stiffness than a decrease in tensile stress in opposite senses in the second flexible link strand of this pair of flexible links, wherein the instrument is actuated at a distance from the body, for instance for cleaning, testing, or the like.

### BRIEF DESCRIPTION OF THE DRAWINGS

Other advantages and characteristics are derived from dependent claims and the exemplary embodiments. The following are shown in a partially schematic manner.

FIG. 1: a portion of a surgical instrument according to one embodiment of the present invention; and

FIG. 2: a portion of a surgical instrument according to a further embodiment of the present invention corresponding to the depiction in FIG. 1.

### DETAILED DESCRIPTION

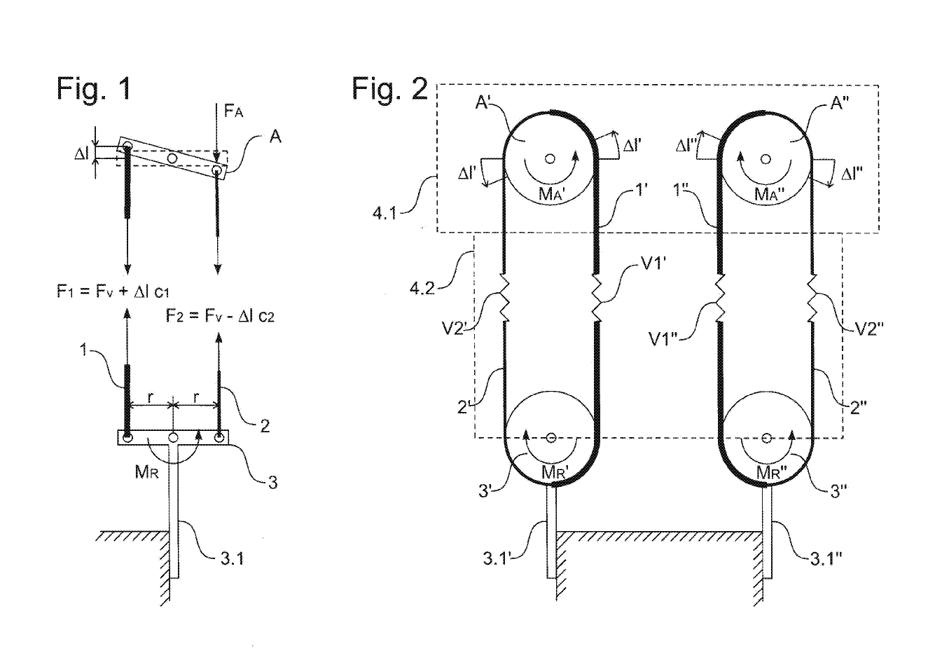

FIG. 1, which was already referenced above, shows a portion of a surgical instrument according to an embodiment of the present invention. The surgical instrument has an input assembly with an input member in the form of a rotatingly seated link arm A and an end effector output assembly with an output member in the form of a further rotatingly seated link arm 3, onto which an end effector 3.1 is attached.

An input and output member are coupled through a pre-tensioned pair of flexible links with a first flexible link strand 1 and a second counter-rotating flexible link strand 2. Due to the pre-tension, pre-tension cutting forces F_{V} act on the Euler cut sections.

FIG. 1 shows a static equilibrium based on applying an input force F_{A} applied by an actuator (not shown) onto the input member A. This force attempts to rotate the end effector 3.1 in a clockwise direction. However, since the end effector contacts an environment, for instance a lumen in the body's interior, the end effector is unable to move—the reactive moment M_{R} acts on it.

In the static equilibrium, the pulling first flexible link strand 1 is elongated by Δl, whereas the pre-tension related elongation of the yielding second flexible link strand 2 is reduced by Δl in a counter-rotating manner. FIG. 1 shows this elongation in exaggerated form for emphasis. Moreover, the elastic deformations of input and output member, end effector and environment resp. lumen are disregarded.

As discussed above, a cut force F₁ is increased in this static equilibrium on the Euler cut section of the pulling first flexible link strand 1, whereas the corresponding cut force F₂ of the yielding second flexible link strand 2 is appropriately reduced.

The first flexible link strand 1 has a geometry-based, in particular cross-section-based, greater tensile stiffness c₁ than the second flexible link strand 2, as indicated in FIG. 1 by different line thicknesses of the first and second flexible link strand. Additionally, or alternatively, the material of which the first flexible link strand consists can also have a greater modulus of elasticity than the material of which the second flexible link strand consists.

As discussed above, the maximum reactive moment M_{R, max.} and therefore the maximum force that can be applied by the end effector 3.1 on the environment and/or the maximum transferrable input force F_{A, max.} pursuant to equation (7) is

M_{R, max}=(c₁/c₂+1)*F_{V}*r

The tensile stiffness c in this case is, for example, the ratio between a test tensile force F and a change in length of the particular flexible link strand effected by said tensile force, in particular relative to its total original length l, which is derived by the cross section area Q and the modulus of elasticity E as

c=(E*Q)/l.

One sees that the greater tensile stiffness c₁ versus c₂—when compared to a homogeneous pair of flexible links with the same tensile stiffnesses—at the same pre-tension advantageously results in a higher maximum reactive moment resp. a lower required pre-tension for the same maximum reactive moment.

FIG. 2 shows corresponding to the depiction in FIG. 1, a portion of a surgical instrument according to a further embodiment of the present invention, wherein elements that correspond to each other are labeled by identical labels, where appropriate by labels differentiated with prime marks (′, ″), so that the aforementioned specification is referenced and the following specification strictly discusses the differences to the embodiment in FIG. 1.

The surgical instrument in FIG. 2 shows two pre-tensioned pairs of flexible links (1′, 2′) (1″, 2″), each with a first flexible link strand 1′ and/or 1″ and a counter-rotating second flexible link strand 2′ and/or 2″ to actuate in opposite senses two degrees of freedom of an end effector in the form of a clamp with two jaws 3.1′, 3.1″ that clamp a lumen between them and therefore experience reactive moments M_{R}′ and/or M_{R}″.

The pairs of flexible links are arranged in a common instrument shaft 4.2, whose half facing the end effector (bottom in FIG. 2) is inserted into a natural or minimally invasive body opening (not shown). The opposite end has a drive assembly with two actuators in the form of electro-motors (not shown) and synchronized with control technology arranged in a housing 4.1, wherein the instrument is coupled to a robot by means of an interface, and the electro-motors are functionally connected with input members A′, A″, for instance directly connected or coupled through a gearbox (not shown). The flexible link strands are separately pretensioned through a multi-part pre-tensioning device with springs V1′, V2′, V1″ resp. V2″.

The input and output members A′, A″, 3′ and/or 3″ in the embodiment in FIG. 2 are configured as drive sheaves around which the pairs of flexible links are each partially wrapped. Accordingly, the first flexible link strand and the second flexible link strand of each pair of flexible links are each connected to each other on the side facing the end effector (bottom in FIG. 2) and on the side facing the input (top in FIG. 2), whereas the first flexible link strand and the second flexible link strand in the embodiment in FIG. 1 are secured separately from each other in the input and output member A, 3. The free sections between the inbound and outbound side of the flexible link from and/or to the particular drive sheave form the flexible link strands in the embodiment in FIG. 2.

In the embodiment in FIG. 2, the closing directions of jaws 3.1′, 3.1″ form the primary working directions of the particular pairs of flexible links. Accordingly, their first flexible link strands 1′, 1″ are arranged to actuate the degrees of freedom of the clamp jaws 3.1′. 3.1″ in the primary working direction, in particular for closing.

### LABEL REFERENCE LIST

1; 1′; 1″ first flexible link strand
2; 2′; 2″ second flexible link strand
3; 3′; 3″ output member (end effector output assembly)
3.1; 3.1′; 3.1″ end effector
4.1 drive assembly housing
4.2 instrument shaft
A; A′; A″ input member (input assembly)
V1′, V2′
V1″, V2″ pre-tensioning device (part)

What is claimed is:

1-15. (canceled)

16. A surgical instrument, comprising:
a first input assembly; and
at least one pair of pre-tensioned flexible links operatively coupled with the first input assembly, the at least one pair of flexible links including a first flexible link strand and a second flexible link strand acting counter to the first flexible link strand, wherein the first flexible link strand has a greater tensile stiffness than the second flexible link strand;
the at least one pair of flexible links operable with the first input assembly to actuate a first degree of freedom of the surgical instrument in opposite senses.

17. The surgical instrument of claim 16, further comprising:
a second input assembly; and
at least one second pair of pre-tensioned flexible links including a first flexible link strand and a counter-acting second flexible link strand, wherein the first flexible link strand has a greater tensile stiffness than the second flexible link strand;
the flexible links of the at least one second pair of flexible links operable with the second input assembly to actuate a second degree of freedom of the instrument in opposite senses.

18. The surgical instrument of claim 16, wherein the tensile stiffness of the first flexible link strand is at least 10 percent greater than the tensile stiffness of the second flexible link strand.

19. The surgical instrument of claim 16, wherein the tensile stiffness of the first flexible link strand is at least 25 percent greater than the tensile stiffness of the second flexible link strand.

20. The surgical instrument of claim 16, wherein the tensile stiffness of the first flexible link strand is at least 50 percent greater than the tensile stiffness of the second flexible link strand.

21. The surgical instrument of claim 16, wherein a material of the first flexible link strand has a greater tensile stiffness than a material of the second flexible link strand.

22. The surgical instrument of claim 16, wherein the first flexible link strand has a greater geometry-based tensile stiffness than the second flexible link strand.

23. The surgical instrument of claim 22, wherein the greater geometry-based tensile stiffness of the first flexible link strand is a cross-section-based tensile stiffness.

24. The surgical instrument of claim 16, wherein at least one of the first or second link strands comprises a first section with a first tensile stiffness and a second section with a second tensile stiffness different than the first tensile stiffness.

25. The surgical instrument of claim 16, wherein the first flexible link strand is arranged to actuate the first degree of freedom in a primary working direction.

26. The surgical instrument of claim 25, wherein first flexible link strand actuates a degree of freedom such that the surgical instruments performs a closing or cutting operation.

27. The surgical instrument of claim 16, wherein at least one of:
the first and second link strands of the flexible links are coupled with an output assembly, and are either separate from one another or are connected together adjacent the output assembly; or
the first and second link strands of the flexible links are either separate from one another or are connected together adjacent the input assembly.

28. The surgical instrument of claim 16, further comprising:
at least one pre-tensioning device configured to jointly or separately pre-tension the first flexible link strand and the second flexible link strand.

29. The surgical instrument of claim 28, wherein the pre-tensioning device is adjustable to pre-tension the first and second flexible link strands.

30. The surgical instrument of claim 16, further comprising an actuator assembly comprising at least one actuator operatively coupled with the input assembly.

31. The surgical instrument of claim 16, further comprising:
an instrument shaft within which the at least one pair of flexible links is arranged, the instrument shaft adapted for insertion through a natural or minimally invasive body opening.

32. The surgical instrument of claim 16, further comprising an interface for coupling the surgical instrument to a robot.

33. A surgical robot, comprising:
a surgical instrument, the surgical instrument comprising:
a first input assembly; and
at least one pair of pre-tensioned flexible links operatively coupled with the first input assembly, the at least one pair of flexible links including a first flexible link strand and a counter-acting second flexible link strand, wherein the first flexible link strand has a greater tensile stiffness than the second flexible link strand;
the at least one pair of flexible links operable with the first input assembly to actuate a first degree of freedom of the surgical instrument in opposite senses.

34. A method for specifying a surgical instrument, the method comprising:
obtaining an input assembly;
operatively coupling at least one pair of pre-tensioned flexible links with the input assembly, the flexible links being operable with the input assembly to actuate a first degree of freedom of the surgical instrument in opposite senses;
wherein the at least one pair of flexible links includes a first flexible link strand and a counter-acting second flexible link strand; and
specifying a tensile stiffness of the first flexible link strand to be greater than a tensile stiffness of the second flexible link strand.

35. A method for actuating a surgical instrument at a distance from a body, the method comprising:
obtaining a surgical instrument, the surgical instrument comprising:
an input assembly; and
at least one pair of pre-tensioned flexible links operatively coupled with the input assembly, the at least one pair of flexible links including a first flexible link strand and a counter-acting second flexible link strand, wherein the first flexible link strand has a greater tensile stiffness than the second flexible link strand;
the at least one pair of flexible links operable with the input assembly to actuate a first degree of freedom of the surgical instrument in opposite senses; and
actuating the input assembly such that an increase in tensile stress in the first flexible link strand is greater than a reduction in tensile stress in an opposite sense in the second flexible link strand.
